(19) United States
(12) Patent Application Publication
(10) Pub. No.: US 2016/0220169 A1
(43) Pub. Date: Aug. 4, 2016

(54) Method and Apparatus for Detecting Seizures Including Audio Characterization

(71) Applicant: BRAIN SENTINEL, INC., San Antonio, TX (US)

(72) Inventor: Michael R. Girouard, San Antonio, TX (US)

(21) Appl. No.: 14/917,880

(22) PCT Filed: Sep. 9, 2014
(86) PCT No.: PCT/US14/54837

§ 371 Date: Mar. 9, 2016

Related U.S. Application Data
(63) Continuation-in-part of application No. 13/275,309, filed on Oct. 17, 2011, now Pat. No. 8,983,591.
Application No. PCT/US14/54837 is a continuation-in-part of application No. 13/542,596, filed on Jul. 5, 2012, now Pat. No. 9,186,105.
(60) Provisional application No. 62/001,302, filed on May 21, 2014.
(60) Provisional application No. 61/979,225, filed on Apr. 14, 2014.
(60) Provisional application No. 61/969,660, filed on Mar. 24, 2014.
(60) Provisional application No. 61/915,235, filed on Dec. 12, 2013.
(60) Provisional application No. 61/875,429, filed on Sep. 9, 2013.
(60) Provisional application No. 61/393,747, filed on Oct. 15, 2010.
(60) Provisional application No. 61/504,582, filed on Jul. 5, 2011.

(51) Int. Cl. A61B 5/00; A61B 7/02; A61B 5/0492

(52) U.S. Cl. CPC A61B 5/4094; A61B 5/0492; A61B 7/02; A61B 5/746; A61B 5/0004; A61B 2562/0204

(57) ABSTRACT

A method of monitoring a patient for seizures with motor manifestations may comprise monitoring a patient using one or more EMG and acoustic sensors and determining whether the collected data is indicative of seizure activity.

### CROSS REFERENCE TO RELATED APPLICATIONS

This application claims priority to U.S. Provisional Patent Application No. 61/875,429 filed Sep. 9, 2013, U.S. Provisional Patent Application No. 61/915,236 filed Dec. 12, 2013, U.S. Provisional Patent Application No. 61/969.660 filed Mar. 24. 2014, U.S. Provisional Patent Application No. 61/979,225 filed Apr. 14, 2014, and U.S. Provisional Patent Application No. 62/001,302.filed May 21, 2014, and is a continuation-in-part of U.S. patent application Ser. No. 13/275,309.filed Oct. 17, 2011, which claims priority to U.S. Provisional Patent Application Ser. No. 61/1393,747 filed Oct. 15, 2010 and is as continuation-in-part of U.S. patent application Ser. No. 13/542,596 filed Jul. 7, 2012, which claims priority to U.S. Provisional Patent Application Ser. No. 61/504.582 filed Jul. 5, 2011.

### BACKGROUND

A seizure may be characterized as abnormal or excessive synchronous activity in the brain. At the beginning of a seizure, neurons in the brain may begin to fire at a particular location. As the seizure progresses, this firing of neurons may spread across the brain, and in some cases, many areas of the brain may become engulfed in this activity. Seizure activity in the brain may cause the brain to send electrical signals through the peripheral nervous system to different muscles the activation of which may initiate as redistribution of ions within muscle fibers. In electromyography (EMG), an electrode may be placed on or near the skin and configured to measure changes in electrical potential resulting from ion flow during this muscle activation.

EMG detection may be particularly amenable for use in apparatuses that may be minimally intrusive, minimally interfere with daily activities and which may be comfortably used while sleeping. Therefore, methods of monitoring the seizure activity of patients, including methods for monitoring in ambulatory or home settings, may benefit from the use of EMG detection. For some patients, a seizure event may also be presented as an audible scream or vocalization which may typically occur at the start of a seizure. Like EMG detection, audio detection of seizures may be particularly amenable to methods of patient monitoring that may be minimally intrusive, and monitoring of seizure activity using one or more acoustic sensors individually or in combination with EMG may be used in improved methods of monitoring a patient for seizure activity.

### SUMMARY

In some embodiments, a method of monitoring a patient for seizures with motor manifestations may comprise monitoring a patient using one or more EMG and acoustic sensors and determining whether data collected using either sensor type exceeds a threshold value. In some embodiments, if a threshold value is met, a patient may be further monitored for a subsequent period of time and an alarm protocol may be initiated if a corroborating event or second threshold is reached during that subsequent dine period.

### BRIEF DESCRIPTION OF THE DRAWINGS

FIG. 1 illustrates one embodiment of a seizure detection system that includes one or more acoustic sensors.

FIG. 2 illustrates one embodiment of a method of monitoring a patient using data collected or received from an acoustic sensor.

FIG. 3 illustrates a further embodiment of a method of monitoring a patient using data collected or received from an acoustic sensor.

FIG. 4 illustrates a method of monitoring a patient using data collected or received from an acoustic sensor and that may be used together with EMG data.

FIG. 5 illustrates another method of monitoring a patient using audio data that may be used together with EMG data.

### DETAILED DESCRIPTION

The apparatuses and methods described herein may be used to detect seizures and timely alert caregivers of seizure-related events and may further be used to provide early indication that a detected seizure event may pose certain risks of adverse effects including SUDEP. The apparatuses may include sensors attached to a patient or patient's clothing and may be configured for measurement or muscle electrical activity using electromyography (EMG). Detection of seizures using EMG electrodes and/or other sensors is further described, for example, in Applicant's U.S. patent application Ser. Nos. 13/275,309 and 13/542,596 and Applicant's U.S. Provisional Patent Application Nos. 61/875,429, 61/910,827, 61/915,236, 61/969,660, 61/979,225, and 62/001,302 the disclosures of each of which are herein fully incorporated by reference. As described herein., an acoustic sensor may further be used to monitor the state of a patient, and in some embodiments, audio data may be collected or received from an acoustic sensor and/or stored along with EMG data. Audio data may be used to enhance the accuracy of real time seizure detection and/or used in review of collected sensor data. For example, audio data may be collected, analyzed in real-time, and used in making a decision about whether to alert a caregiver that a patient may be experiencing a seizure. In some embodiments, audio data may be used to corroborate the detection of seizure activity based on one or more portions of EMG data, including EMG data collected during one or more early or pre-seizure time periods, and may, in combination with the EMG data, be used to initiate an emergency or other alarm response. Collected audio data may also be analyzed at times after a period of monitoring and may be used to verify whether a seizure or seizure related event has occurred.

In some embodiments, monitoring a patient using collected or received audio data may be either semi or fully automated. For example, a monitoring system may be configured to operate without the need for interpretation by a remote caregiver using a processor configured to analyze the data for features characteristic of seizure activity. In some embodiments, a processor may be configured to identify repetitive patterns included in audio data that meet one or more criteria that may be indicative of a seizure and weigh the presence of those patterns in a method that may be used to trigger an alarm or initiate another system response. And, those methods may be automated without need for caregiver input or interpretation. Alternatively, audio data may be transmitted to a remote caregiver for interpretation. Particularly, data suspected of being related to seizure activity may be sent to a caregiver for review after initial identification or screening using an automated program. For example, a processor may be configured to identify patterns associated with seizure activity and if those patterns are found present, audio data may be transmitted to a caregiver for further interpretation and/or verification of seizure activity. Therefore, a processor may be configured to directly trigger an alarm using one or more algorithms that include audio data or may be configured to filter sounds from other sound features identifying those most likely to indicate the presence of a seizure.

In some embodiments, audio data may be processed in order to calculate one or more input values for use in a seizure detection algorithm. A detection algorithm that incorporates audio data may operate individually or in combination with other data to detect a seizure. For example, in some embodiments, audio data may be input into a monitoring routine that also includes inputs derived from one or more EMG and/or other sensors. In some of those embodiments, an audio detection routine may focus on one type of seizure or a particular manifestation of one or more seizure types. For example, a patient experiencing a seizure may sometimes produce characteristic sounds indicative of respiratory stress, but for other seizures, the patient may fail to produce that particular sound pattern. An audio detection routine may be configured to be selective the one or more particular manifestations of seizure activity and when identified confidence in detection may be high. However, in some embodiments, it may be beneficial to combine audio detection with other sensor data particularly including EMG which may be made highly responsive to generalized seizure activity. And, in some embodiments, audio detection may be combined with EMG not only to improve detection efficiency but also to help classify identified seizures. In some embodiments, more than one audio detection routine may be run together in a method of analyzing data for various audio signatures that may be present for different seizure manifestations. For example, in some embodiments, one audio detection routine may examine audio data for the presence of a high amplitude signal that may indicate a scream or examine audio data for a high amplitude signal followed by a sustained portion of lower audio amplitude and a second audio detection routine may examine audio data for one or more patterns and determine if the patterns show periodicity indicative of one or more parts of a seizure. Those routines may, in some embodiments, be patient specific and tailored to detect sounds particular for a given patient or patient demographic. And, in some embodiments, voice recognition software may be used to identify that a given sound was derived from a certain patient.

Audio data may, in some embodiments, be collected or received during one or more time periods and characteristics of the data calculated over time. For example, a characteristic derived from audio data may be a metric related to the strength or power of a sound wave from which the data was derived such as a signal amplitude or amplitude as compared to a reference level and a value for the characteristic may be expressed, for example, in decibels or another relative unit expressing amplitude, strength, or power of a sound wave. A characteristic of audio data may be tracked and trends in the data may be analyzed for seizure characteristics. For example, a characteristic such as signal amplitude may be considered over time and the presence of one or more data patterns or key points in the signal (such as local maximum values or local maximum value meeting some threshold amplitude may be determined). A local maximum value may be related to a particular physical activity executed by the patient (such as gasping of air) and may repeat. For example, each time the patient executes the activity a local maximum value or local maximum value meeting some threshold amplitude may be present. By tracking the position of local maximum values or other repeating pattern or value the underlying activity executed by the patient may then be monitored. For example, the periodicity and/or duration of intervals of time of or between repetitive patterns of audio data may be determined and compared to those typical for a patient experiencing a seizure. As used herein, the term “periodicity” refers to how regular a certain pattern may manifest or repeat over time. In some embodiments, one or more characteristics of audio data may be determined and used to identify one or more repetitive data patterns. Characteristics of audio data may, by way of nonlimiting example, include audio signal intensity or amplitude, amplitude at a given frequency (or over a certain frequency range), rate of change of amplitude, spectral slope, other data, or combinations of audio characteristics thereof. In some embodiments, data from a collected or received signal may be compared to one or more model patterns of data associated with an activity that may typically repeat for a patient experiencing a seizure. For example, using pattern recognition software similarity of data to a model pattern may be determined (such as by using regression analysis), and a certainty value for whether a given portion of data match the pattern may be determined. A certainty that a detected pattern corresponds to an activity executed by a patient during a seizure may be determined and to increase the confidence that data may properly be identified as related to a seizure trends in the pattern over time may be determined. For example, when a patient is under respiratory stress they may tend to gasp repetitively over time, but as the patient tires sound produced during gasping may weaken or shift in frequency. When examining collected or received data expected to match a patient activity (such as a gasp) changes and/or shifts in the data may be compared to those typical for a patient experiencing a seizure (during normal or abnormal seizure progression) and if those changes and/or shifts are within expected bounds certainty of seizure detection may be improved.

In some embodiments, to identify a repeating pattern in collected or received audio data one or more algorithms may be executed to compare data to a model set of data derived or recorded from one or more actions executed by a patient during a seizure and a certainty value may be assigned to an identified portion of data such as using one or more data regression algorithms. For example, collected data and model data may be overlaid (varying the relative position of a set of clinical data and model data), and in some embodiments, a point-to-point analysis of deviations (for each varying position) may be executed and when overlaid as appropriate to minimize the deviations a similarity value between the clinical patient data and model data may be determined. If the overall deviation between points is suitable a pattern may be deemed to be detected. To further relate the pattern in seizure activity, a periodicity value of a plurality of detected putters may then be determined. In some embodiments, data may also be processed by one or more algorithms to identify that the sound is related to a patient. An algorithm to identify that a sound is related to a patient may, for example, include or be based on any of various yoke recognition algorithms or programs.

In some embodiments, audio data may be filtered and/or corrected to account for ambient noises or a level of ambient noise, and in some embodiments, spatial filtering of an audio signal may be used to isolate sounds originating from different locations within or near a region of monitoring. In some embodiments, audio data may be classified based one or more events that may produce a certain sound or sound component. For example, audio data may be classified as being characteristic of any number of events including by way of nonlimiting example occurrence of a seizure, human speech, shutting of doors, barking of a dog, walking, ringing telephone, other events, and combinations thereof. Some events may be deemed background noise that may not indicate the presence of a seizure. That is, non-seizure related sources of noise may be characterized in some embodiments, events that may be indirectly produced by a patient during a seizure may be characterized. For example, during a cloak-portion of a seizure, a patient may move back and forth causing oscillation of nearby objects, such as furniture, which may produce an audible sound. And, in some embodiments, an object such as an item of furniture may be purposefully modified to produce a characteristic sound when moved in a rhythmic manner. For example, a bell or other sound device may be associated with an item of furniture that produces a characteristic sound in response to nearby movement. Preferably, that bell may produce an oscillation that is accurately captured by an acoustic transducer the oscillation being different than other sounds. For example, a sound making device may oscillate at a frequency that is readily passed by an inverse notch or combination of high pass and low pass filters. In some embodiments, to facilitate classification of audio data, sounds may be characterized in terms of intensity, spectral shape or other characteristics and stored in a database for comparison to data collected during monitoring. Collected data and/or spatially filtered data may be fit to data derived from one or more known sounds and a probability that a sound or component of a total sound may be provided from a seizure (or discounted as associated with a non-seizure event) may then be calculated and used in a seizure detection algorithm.

In some embodiments, audio data may be collected using one or more monitoring routines that may run intermittently or that may be configured to trigger certain responses only if activated by being preceded within a time period by other events. For example, audio data may, in some embodiments, be collected, but may only initiate an alarm response if the audio data is temporally correlated with the detection of EMG data associated with a seizure related event. For example, some routines for electromyography may examine whether a patient may be experiencing weak motor manifestations typically present prior to a seizure. And, if those routines produce a response, it may be deemed that the patient is at risk of having a seizure. In some embodiments, weak detections may terminate passively without interrupting the patient or produce an active response if, for example, the weak events fail to terminate or if the detection is corroborated by another event. In some embodiments, corroboration of initial motor manifestations of a seizure, including manifestations detected prior to or without a clonic phase portion of a seizure, may be made based on one or more detected audio patterns. That is, in some embodiments, an audio detection routine may be executed or activated to provide a given response only if preceded by a detection of prior EMG data. For example, if weak motor manifestations are detected with EMG, an audio detection routine may become active such that the routine may issue an alarm if the audio data indicates the presence of seizure activity and corroborates the EMG data. Because those weak motor manifestations may only be present intermittently—whether a seizure actually manifests or not, the probability of inadvertent or false-positive initiation of an alarm based on collected audio data may be minimized.

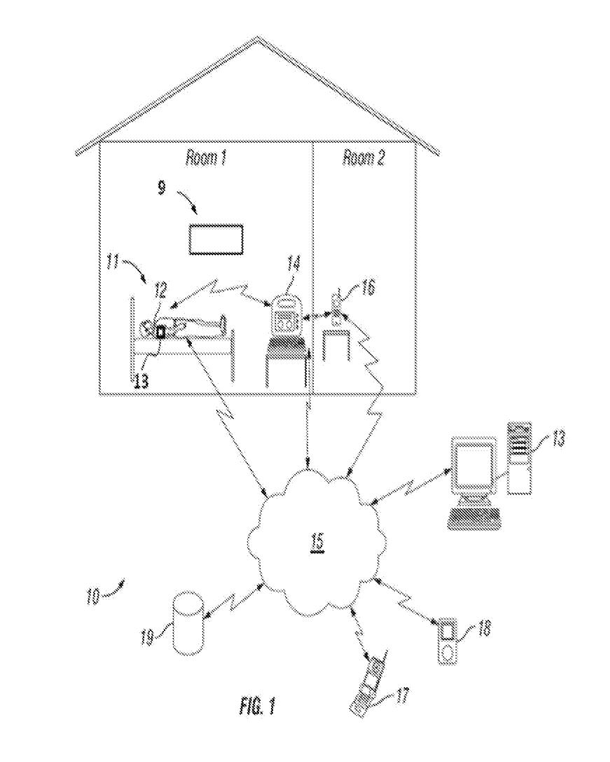

A variety of systems may be suitably used for collecting EMG, audio, and other patient-related data, prioritizing data for storage, organizing such data for system optimization, and/or initiating an alarm in response to a suspected seizure. FIG. 1 illustrates an exemplary embodiment of such a system. In the embodiment of FIG. 1, a seizure detection system 10 may include a video camera 9, a detection unit 12, an acoustic sensor 13, a base station 14, and an alert transceiver 16. The detection unit may comprise one or more EMG electrodes capable of detecting electrical signals from muscles at or near the skin surface of a patient, and delivering those electrical EMG signals to a processor for processing. The base station may comprise a computer capable of receiving and processing EMG signals from the detection unit and/or acoustic data from an acoustic sensor, determining from the processed EMG and/or acoustic signals whether a seizure may have occurred, and sending an alert to a caregiver. An alert transceiver may be carried by, or placed near, a caregiver to receive and relay alerts transmitted by the base station. Other components that may be included in the system 10, including for example, an alert transceiver 16, wireless device 17, 18, storage database 19, and one or more environmental transceivers (not shown) are described in greater detail in Applicant's U.S. patent application Ser. Nos. 13/275,309 and 13/542,596.

As shown in FIG. 1, one or more acoustic sensors 13 may be included in a detection system 10. Acoustic sensors may, for example, be placed at one or more locations within or near a monitoring area. An acoustic sensor may, in some embodiments, be attached to a patient or patient's clothing. Therefore, an acoustic sensor may be attached and may move along with a patient or may remain stationary as a patient moves. In FIG. 1, acoustic sensor 13 is shown to be a separate unit from other elements. For example, a detection unit 12 may be attached to one arm of a patient and an acoustic sensor 13 may be worn on the same or other arm. However, an acoustic sensor may also be integrated into one or more other devices. For example, an acoustic sensor may be integrated into any of video camera 9, detection unit 12, base station 4, or integrated in some other device or element.

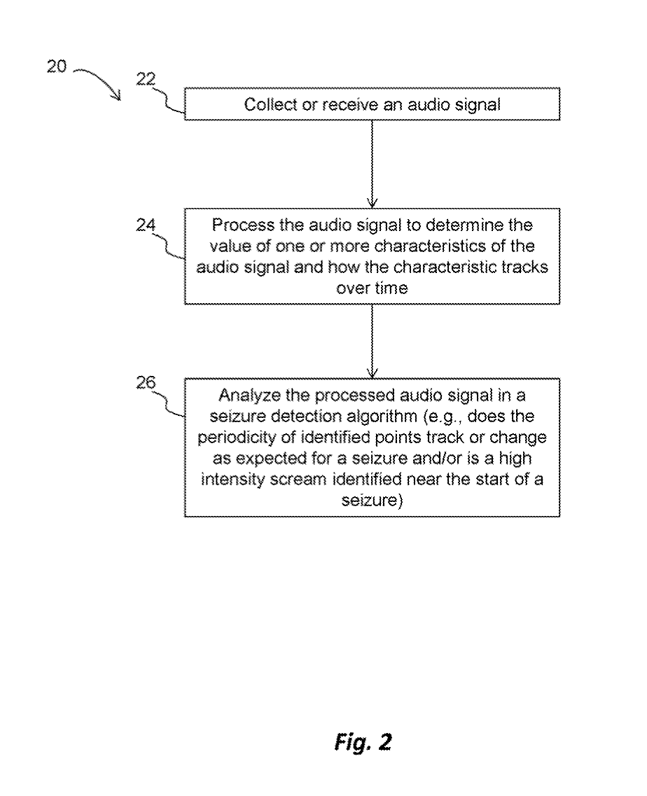

FIG. 2 illustrates an exemplary method 20 of analyzing an audio signal for seizure characteristics. In a step 22, an audio signal may be collected using one or more acoustic sensors or data may be imported into a processor for analysis. An acoustic sensor or microphone may, for example, include an acoustic-to-electric transducer suitable for converting a sound wave into an electrical signal. A transducer may, in some embodiments, operate without significant signal distortion over a desired frequency range which may, for example, include the frequency range of human speech and/or include other frequencies such as may be useful to spectrally characterize any of various sources of environmental noise or sound producing devices including those that may be specifically associated with one or more units of furniture or objects in a monitoring locale. As used herein, spectral characterization of acoustic data refers to description of signal intensity over one or more frequencies. In the step 24, a collected or received audio signal may be processed to determine the value of one or more characteristics of the audio data. For example, in the step 24, signal may be processed or conditioned such as to remove background noise and/or to isolate a desired frequency band or distribution of frequency bands. In some embodiments, signal may be processed through an analog-to-digital converter suitable for processing of signals that may be as high as about 5 KHz to about 10 KHz. In some embodiments, one or more high and/or low pass filters may also be used to condition a collected audio signal.

Processing may, in some embodiments, further include comparison of signal to audio data previously acquired during one or more reference periods. For example, a reference period may be collected, and baseline audio characteristics of the reference period such as a baseline level of an audio characteristic and/or noise fluctuations in an audio characteristic mar be established. Audio signal collected may, in some embodiments, be processed by scaling a characteristic of audio data in terms of a ratio to a baseline value or scaling in terms of a number of standard deviations above a characteristics baseline noise level. For example, amplitude of audio data or amplitude over one or more frequency bands may be a characteristic that may be compares to baseline amplitude levels and/or otherwise scaled by comparison to a baseline levels of amplitude.

Processing of data in the step 24 may be used to determine the value of one or more characteristics of audio data. For example, in some embodiments, processing of data may be used to assess how a characteristics of audio data, such as its amplitude, tracks over time. For example, in some embodiments, processed audio data may be amplitude data associated with a desired portion of monitored frequencies, and in some embodiments, amplitude data may include all or a selected portion of collected frequencies.

Upon processing of data to determine characteristic values for the data and how the values tracks over time an algorithm may further examine whether characteristic value change over time in a manner expected for seizure activity. For example, in the step 26, in some embodiments, processed data may be analyzed to identify distinct points among the determined values for the characteristic, and examine whether the distinct points meet one or more periodicity requirements associated with seizure activity. For example, a distinct point may be identified if the point meets a threshold amplitude value, and the timing or periodicity between those points may then be examined. That is, step 26 may include comparing data values for a characteristic tracked over time (as describe in step 24), identifying distinctive or critical points based on meeting a threshold criterion and determining if the timing between distinct or critical points over times meets a periodicity requirement.

In some embodiments, a plurality of distinct points may be assessed and periodicity values for times between the points may be determined. However, some trends in an audio signal may not repeat. For example, in some seizures, an initial or high intensity scream (as further described below) may be present and, in some embodiments, an high intensity scream (sometimes followed by a sustained period of lesser amplitude signals) may be identified by analyzing processed audio signal. And, while in some embodiments, audio signal may be input together with other sensor data (preferably EMG data) to detect a seizure, in other embodiments, one or more characteristics of audio signal may be used to directly trigger an alarm. For example, if an audio signal is collected or received (step 22) and if amplitude is tracked over time (step 24) and in analysis of amplitude trends (step 26) signatures of a high intensity scream followed by a delay period and then a repeating series of distinct points or patterns indicative of a plurality of gasps is detected confidence in seizure detection may be high.

In some embodiments, processing and analysis of audio signal may include running one or more pattern recognition programs to identify within audio data if a certain portion of the data matches a pattern. For example, in some embodiments, a distinctive or critical point (as described above) may be a part of a pattern including, for example, a pattern modeled after an activity commonly executed during a seizure. In some embodiments, pattern recognition may include smoothing a set of data, identification of one or more extreme values in a data set, and applying one or more procedures including overlay and regression analysis. For example, a program may identify a local maximum value in an audio data set and attempt to fit data around the local maximum to one or more model functions associated with a certain sound. For example, a model sound may represent or be derived from a recording of a patient gasping for air and a given set of data may be compared to the model sound by overlaying and fitting collected data using regression analysis and determining if the sound meets a threshold level of similarity to the model sound. For example, an algorithm may determine if a certain portion of data matches a pattern of a gasp or matches the pattern of a gasp at some probability.

During a seizure some patients may shout, grunt, or gasp and the overall amplitude or intensity of a resulting acoustic signal may be high. The presence of a spike or sustained spike in acoustic sensor amplitude may therefore tend to correlate with a seizure state. However, other events may also tend to produce high amplitude audio signals. Therefore, in preferred embodiments, processed signal may be analyzed in order to discriminate acoustic data from non-seizure sources. In various embodiments described herein, discrimination of acoustic data from non-seizure events may be achieved in various ways.

For example, when some patients experience a seizure the patient may force a large amount of air through their throat and an audible signal may tend to be produced. Some patients may tend to take in and expel air from the lungs in a repetitive manner, and a resultant sound pattern, sometimes characterized as a grunt or gasp, may be repeated in time with a degree of regularity. Some embodiments herein may analyze a collected audio signal for the presence of a sound pattern that resembles as seizure grunt or gasp. Furthermore, some embodiments may determine if the sound pattern is repeated, and a repeating sound pattern may be used to detect the presence of a seizure. Particularly, the periodicity of a sound pattern of a seizure may be more regular and/or may, for some seizures, include a lower frequency component than some other sounds including for example normal human speech. For example, normal human speech may tend to have more variation than sounds produced during a seizure. Moreover, the regularity of sounds produced in a seizure may be more random in human speech and generally not vary in the same manner as someone who may, for example, be struggling to take in and expel air repetitively as in certain parts of a seizure.

The repetition rate of individual members of a repeating sound pattern for a patient experiencing a seizure may be characterized, and for some patients the number of pattern members present over time may be about 0.5 to about 5 member patterns per second. For example, for some patients at least about three members of a repeating sound pattern for every second may be present at the start of one part of a seizure with the number typically dropping during the seizures progression. That number may drop steadily through a seizures progression or terminate abruptly. That progression may be characterized over time and communicated to a caregiver and may be compared to models of progression including those for normal and abnormal seizure progression or recovery. In some embodiments, the periodicity of a repeating sound pattern may be determined for an individual patient or estimated for a patient based on one or more patient characteristics (e.g., patient age, gender, height, and/or weight), and in some embodiments, an expected periodicity of a seizure sound patters may be estimated prior to patient monitoring.

In some embodiments, sound may be collected and a pattern recognition algorithm may probe resulting acoustic data for one or more distinguishing patterns. For example, sound may be collected and processed to identify portions of audio data associated with a repetitive seizure sound. A distinguishing pattern may be identified based on the presence of a certain data feature or combination of data features. For example, the presence of a threshold local maximum amplitude, threshold local maximum amplitude followed by a sustained period of decreasing acoustic amplitude, or threshold local maximum with surrounding portions similar to one or more model functions may be used to identify a pattern. To identify a pattern, audio data may be binned and integrated over time units (or bins) to improve signal to noise. The data may be binned within periods of time as may be appropriate to track relevant changes through a period of lime such as during inhalation and/or exhalation during a seizure grunt or gasp. For example, in some seizures, audio data from a grunt may change more slowly as one is taking in air and more rapidly as the diaphragm forces air out of the lungs. Some patients may tend to make a recognizable sound near times following when air has been mostly pushed out of the lungs. For example, the patient may gasp to try and catch their breath. And, to reliably capture sounds produced during contraction and/or expansion of the lungs data may, for example, be binned and integrated over periods of up to about 50 milliseconds. A repeating sound pattern may, in some embodiments, be broken up into various parts and individual parts of the sound pattern may be identified. For example, during inhalation and exhalation different sounds may be made and by examining audio data for a characteristic pattern associated with inhalation followed by exhalation abnormal sounds associated with a seizure may be identified. For example, because normal breathing may show a more symmetric profile of inhalation and exhalation than some seizures, breaking up a sound into a first pattern associated with inhalation and a second pattern associated with exhalation may be used in algorithms for detecting the presence of a seizure. That is, the relative time in which a patient is deemed inhaling and exhaling may be identified and a ratio of inhalation time to exhalation time may be determined. A ratio that is significantly different than about 1:1 (such as outside of a range extending from about 0.8:1 to about 1.2:1) may be used to characterize respiratory stress and possible seizure activity. Particularly, in some embodiments, a detected sound may be examined for characteristics of a seizure grunt or gasp, which may include breaking up the data and looking for parts of data typical of inhalation and typical of exhalation and characterizing whether the duration of the parts are more or less symmetric in duration. That is, for struggled breathing, temporal asymmetry with one part lasting longer than the other may be identified.

An algorithm may further determine whether an identified data pattern maintains an expected periodicity. For example, while portions of a grunt may Show asymmetry between inhalation and exhalation parts the overall pattern of inhalation and exhalation may be characterized as having higher regularity than other sounds including speech. For example, if a pattern is present and repeats over time with a regularity of about once every 0.2 to about 2 seconds, and the pattern is detected a number of times such as at least 4 to about 10 times) or over a certain period initiation of a seizure alarm may be encouraged. Any of various points within a detected pattern may be used to identify timing at which a detected pattern occurs and may further be used to assess the periodicity of the pattern. For example, the start, middle or ending time of a detected pattern may be used. Most patterns described herein may include a local maximum amplitude value that meets some threshold and the time of that value may be conveniently used to identify the position in time of a detected pattern.

In some embodiments, changes in periodicity over time may be tracked (even after an alarm may be initiated), and for example, an algorithm may look for signs of abnormal recovery from a seizure. The periodicity of a repeated sound pattern may farther, in some embodiments, be compared to the periodicity of EMG data bursts. For example, both EMG data bursts and periods of respiratory stress may be related to the presence of uncoordinated signals sent from different parts of the brain and for some patients the phase and/or periodicity of bursts and the phase and/or periodicity of audio data produced during periods of respiratory stress may be related and/or tracked together including to identify when a patient may be showing abnormal signs of seizure progression and/or recovery.

In some embodiments, audio data may possess high amplitude (often associated with characteristic frequency changes) during times of a grunt or gasp right after exhalation begins. More generally, any point or points in a pattern including for example points identified as meeting a threshold requirement or condition or other distinct characteristic may be identified and used in a calculation of periodicity. For some patients, during some portions of a seizure a characteristic grunt may be high in amplitude and the patient may repeat a similar sound, but muscle fatigue may dampen the overall amplitude of the sound pattern. That is, a repetitive pattern may be identified some number of times but later repeats may be characterized as having lowered amplitude. Likewise, for some patients one or more periodicity values may drift over time. Therefore, in some embodiments, detection of a characteristic pattern in audio data accompanied by a dampening of overall signal amplitude and/or trends in periodicity may be used in a seizure detection algorithm.

In some embodiments, audio data may be collected and analyzed over a plurality of time intervals. For example, audio data may be analyzed over time intervals as appropriate to capture amplitude and/or frequency changes that may occur during the course of a seizure. For example, in some embodiments, audio data may be divided into intervals of about 0.01 to about 0.1 seconds. During any given interval one or more characteristic value of audio or processed audio data may be calculated and the characteristic value(s) may be stored. An algorithm may analyze characteristic values from successive collection intervals or analyze smoothed data over a period of time and look for one or more characteristic patterns. Upon identification of two or more repeating pattern members, an algorithm may determine whether the pattern meets one or more periodicity requirements for a seizure. For example, a pattern may be identified by meeting a threshold condition such as the presence of a threshold acoustic amplitude value or threshold acoustic amplitude that is a local maximum, and a method may determine a time interval between detected patterns. For example, a time interval between adjacent detections of two threshold amplitude values may be determined. If the time period between the threshold values is characteristic of a seizure state an alarm may be sent or an alarm may be sent if corroborated by other data.

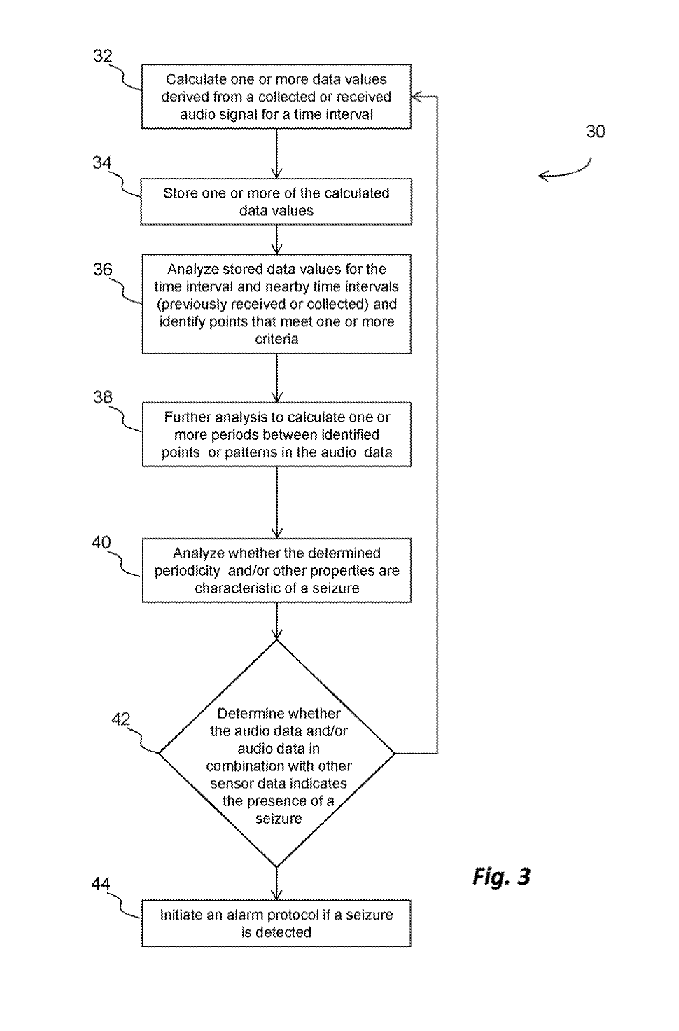

A method 30 of monitoring a patient for seizure characteristics based on the periodicity of one or more distinctive points or characteristic patterns identified from art acoustic signal is illustrated in FIG. 3. In a step 32, signal or suitably processed signal (e.g., filtered or background corrected signal) may be collected or received for a time interval and one or more data values may be calculated from the collected acoustic or audio signal. Data values calculated for an interval may include, by way of nonlimiting example, amplitude data and, in some embodiments, the amplitude data may be associated with one or more spectral frequencies. For example, a patient gasping for air may tend to produce sounds in one or more frequency bands and in some routines for analysis of audio data amplitude data may be isolated based on recorded frequencies for a patient or certain patient demographic.

In a step 34, calculated data value(s) may be stored, and in a step 36 stored data values including data from other nearby intervals may be analyzed to identify data that meet one or more criteria. As described above, in some embodiments, one or more pattern recognition programs may be executed on a set of data over time (e.g., data associated with a number of adjacent time intervals). In some embodiments, if an amplitude of an audio signal in a time interval exceeds a certain threshold or if an audio signal is greater in amplitude than other amplitudes in nearby time intervals (e.g., if the audio signal qualifies as a threshold local maximum value) the acoustic data may satisfy a threshold amplitude criterion. The point may be deemed distinctive and used in further calculations. Other distinct or threshold points may also be identified. For example, in some embodiments, a local minimum in amplitude or an inflection point in amplitude derivative data may be identified. More generally, in some embodiments a distinctive or identified point may be any point in a detected pattern such as the start, middle, or end of a detected pattern that may reliably time stamp when the pattern was detected.

For some patients, acoustic data may be characterized by changes in spectral characteristics. For example, during one portion of a seizure period, such as during initial portions of a grunt, the average frequency of data may be different than the average frequency in other seizure periods such as later portions of the grunt. That is, the dominant frequencies of sounds produced by a patient during a seizure may change, and in some embodiments, a detection algorithm may identify if the frequency distribution of acoustic data changes in a defined manner to meet a criterion. For example, a grunt or gasp may extend over multiple time intervals and in each interval an average or median frequency of signal data may be determined. The average frequency may change over the time period of a grunt and for some, patients may, for example, move to higher frequencies and then to lower frequencies over time. Therefore, a data value calculated in a step 32 may be the average or median frequency value of signal collected during an interval. The data may be stored in a step 34 and compared to other frequency values in nearby intervals in a step 36. For example, if data in an interval is at a point where the average frequency transitions between increasing to decreasing or transitions from decreasing to increasing the time interval may be marked. In some embodiments, a method may determine whether a threshold average or median frequency or local average or median frequency is reached.

In the step 36, data may be analyzed to determine whether a pattern or distinctive point is present in the audio data. For example, a distinctive point may be identified based on meeting one or more criteria such as meeting criteria as a to maximum amplitude value or local maximum amplitude value meeting some threshold. In the step 38, the periodicity of a plurality of identified patterns or points over time may be examined.

In a step 38, one or more times between identified points of a detected pattern may be determined. For example, it may be determined that a 0.5 second period of time elapsed between data intervals identified as meeting a certain threshold because the points satisfy the condition of being threshold local amplitude maximum values. In a step 40 an algorithm anal analyze whether the times are indicative of a seizure. For example, in some embodiments, a time period may be identified as indicative of a seizure if the period is between about 0.2 to about 2 seconds. An algorithm may be tuned so that any number of suitable time periods must be identified before a seizure is indicated. For example, the period between 2 or more identified points or detected patterns may be determined, and as a greater number of suitable periods are measured the algorithm may indicate a higher probability that a seizure may be occurring. For example, in some embodiments, an algorithm may initiate an alarm until at least about 4 to about 10 patterns are identified. The regularity of duration or regularity of time periods may further be analyzed in an algorithm. For example, a standard deviation or other statistical metric associated with multiple periods may be used to analyze whether the determined periods are suitably periodic.

By way of example only, if over a monitoring period a patient inhales and exhales 10 times and if at times near when the patient begins a cycle of inhalation air being carried into the lungs a recognizable sound is produced that sound may be characterized such as by amplitude and or frequency (e.g., a part in the cycle of inhalation and exhalation may be picked out or detected from other points) and identified as a point in a seizure related pattern. With 10 cycles there may be 9 periods between identified points (which in this example is a recognized sound produced during inhalation as a patient gasps for air). That recognized sound may, for example, include a local maximum in amplitude at a certain time or may be characterized in other ways. For example, the times identified may conveniently be characterized by subscripts as follows:

T₁, T₂, T₃, . . . , T₁₀

Relative periods between the identified times may then be calculated as follows:

T₂−T₁=ΔT₁

T₃−T₂=ΔT₂

T₁₀−T₉=ΔT₉

And, any of various procedures may then be used to determine one or more metrics of how periodic or regular in time the periods may be. For example, in one embodiment, time periods between identified points may be determined (as above) and an average time period may then be calculated. The average time period may be compared to individually measured time periods (e.g., how much deviation from the average period is present) and a standard, relative, or percentage deviation then determined. For example, a processor may execute calculations as follows:

$$Average time period = ( Δ {T}_{1} + Δ {T}_{2} + Δ {T}_{3} + … + Δ {T}_{9} ) / ( N = 9 ) = Δ {T}_{( ave )}$$

$$\begin{matrix} Average Deviation = Σ \vert Individual Deviations \vert / No . Deviations \\ = \vert ( Δ {T}_{1} - Δ {T}_{( ave )} ) \vert + \vert ( Δ {T}_{2} - Δ {T}_{( ave )} ) \vert + … \\ \vert ( Δ {T}_{9} - Δ {T}_{( ave )} ) \vert / ( N = 9 )\end{matrix}$$

$$Percentage deviation = [ Average Deviation / Δ {T}_{( ave )} ] \times 100 \%$$

A percentage deviation may, for example, be compared to one or more threshold values of percentage deviation, and if the percentage deviation meets the threshold criteria, periodicity of the detected pattern (e.g., series of 10 inhalation and exhalation producing 10 repeating patterns in the above example) may be viewed as indicative of seizure activity. For example, if the periodicity requirement is fulfilled then an alarm or other response may be executed. An algorithm may, in some embodiments, include comparison of a percentage deviation to one or more threshold values including a minimum percentage deviation and/or a maximum percentage deviation. For example, a repeating noise source that is artificially periodic may show very low percentage deviation and may not be deemed indicative of a seizure. However, human speech which may be more random than sounds made during a seizure may be less periodic. And, in some embodiments, an audio detection method may include comparison of data to both a minimum and/or maximum percentage deviation (or other suitable metric of periodicity) and comparison to a minimum and/or maximum period. For example, where a portion of audio data has a pattern that repeats within threshold for percentage deviation (e.g.. meeting minimum and maximum thresholds for periodicity) and where the portion of audio data includes a pattern that repeats between some minimum and maximum number of times per second the audio data may be deemed indicative of a seizure.

In some embodiments, acoustic data may be used individually to trigger an alarm state. However, in some embodiments, a detection algorithm may also analyze (as shown in a step 42) whether other sensor data (e.g., EMG data) supports a finding that a seizure may be present. For example, if acoustic data is collected and it is determined that the data is characteristic of a seizure and in the same time period threshold EMG values area also satisfied a method 30 may deem certainty of seizure detection to be high and may initiate an alarm protocol in a step 44. In some embodiments, acoustic data may be weighted together with EMG data to determine the likelihood that a seizure may be present. And, in some embodiments, acoustic data may be used to corroborate a finding that weak motor manifestations are indicative of seizure activity. In some embodiments, audio data may act as input in a supervisory algorithm as described in Applicant's related co-pending application Ser. No. 13/275,309 filed Oct. 17, 2011 and herein incorporated by reference. For some patients, a temporal delay between audible manifestations of a seizure and muscular manifestations of a seizure may sometimes occur, and a time period in which the EMG and acoustic data are determined to be related may be adjusted accordingly.

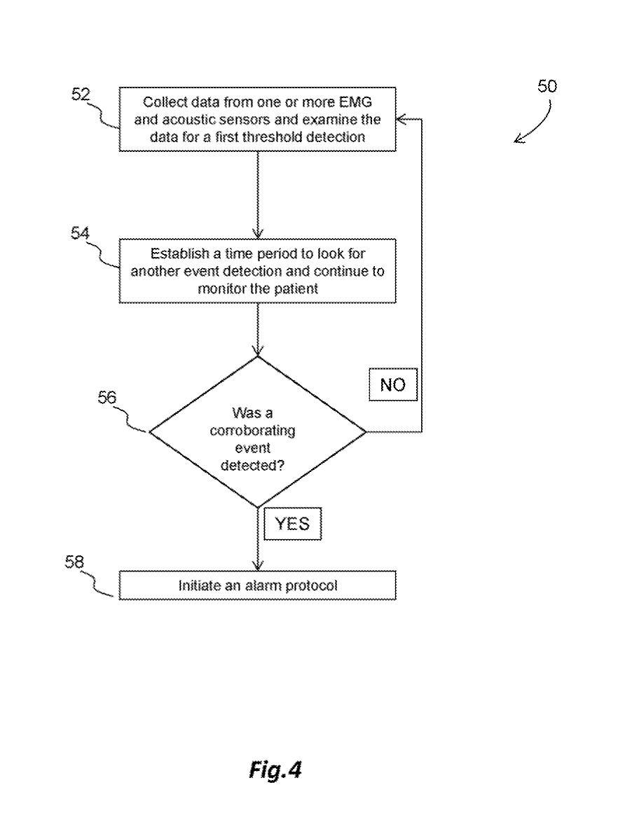

In some embodiments, a seizure detection algorithm may include inputs from each of one or more EMG sensors and one or more acoustic sensors, and for example, if sensors of both types exceed appropriate threshold levels an alarm state be triggered. Some of those embodiments may monitor the periodicity of detected acoustic patterns and/or may integrate other signatures of acoustic data. FIG. 4 illustrates an exemplary method 50 wherein an alarm may be initiated if, for example, each of an EMG and acoustic sensor exceed respective threshold levels during a certain time period. In a step 52, a patient may be monitored using a combination of EMG and acoustic sensors, and the method may look for a first threshold detection event. If either sensor exceeds a threshold, the method may, as shown in a step 54, establish a time period for the monitoring of a threshold event of the other sensor type and continue to monitor the patient. For example, if a first event is the exceeding of an EMG threshold the method may establish a period following that event wherein threshold detection of an audio signal may trigger an alarm. Therefore, as shown in a step 56, a method 50 may determine whether threshold detection of both an EMG and acoustic sensor was met within the established time period. If both threshold EMG and threshold acoustic events were satisfied, as shown in step 58, an alarm protocol may be initiated. Alternatively, if no corroborating event was detected, the system may return to monitoring a patient for a next threshold event. For example, a method 50 may require that one event is detected and that a corroborating event is detected within a time period of up to about 2 minutes or up to about 5 minutes.

For some patients, sounds produced during one part of a seizure may be different than produced during other parts of a seizure. For example, for some patients, often times during a tonic portion of a seizure a patient may rapidly exhale sometimes with a loud scream. The patient may not inhale and begin rhythmic breathing for some period of time. For example, during or after onset of the clonic phase the patient may resume inhaling and at some time the patient may begin to repetitively produce a sound pattern often times as they attempt to regain stable breathing. Some methods herein may took at audio data over time and by identifying features typical of various parts of a seizure those features may be analyzed together to increase confidence in seizure detection. For example, a method of monitoring a patient may include analyzing collected audio data for a high amplitude scream or sound typical of the onset of a seizure and then track the data to look for patterns of an attempt to regain stable breathing. For example, if a high amplitude scream is followed by lower amplitude audio signals for some characteristic time and then followed by a repetitive pattern (such as discussed above with respect to FIG. 4), a seizure may be deemed present and an alarm or other response initiated.

Moreover, in some embodiments of methods of detecting a seizure, audio data may be collected along with other sensor data. If trends in the audio data seem to indicate transition between more than one part of a seizure (such as discussed above), and if the other sensor data corroborates those transitions confidence of seizure detection may be greatly improved. For example, in some embodiments, more than one electromyography routine may be executed together with collection of audio signal, and the plurality of data may be used to not only detect a seizure, but to also to track changes in seizure activity during transition between one or more seizure phases. Various applications associated with the treatment or termination of seizures (e.g., such as may include Vagal nerve stimulation), selective collection or transmission of additional sensor data, and/or selective and customized responses to a detected seizure condition may benefit from the detection and tracking of changes in seizure activity as described herein.

In some embodiments, a method of monitoring a patient for seizure activity may include a first EMG routine that is highly responsive to initial motor manifestations and/or tonic activity and a second EMG routine may be selective for clonic-phase activity. Routines that may be made responsive or selective for detection of initial motor manifestations typical of seizure activity or for different phases of a seizure are, for example, described in Applicant's Co-pending Provisional Application No. 62/001,302 filed May 21, 2014 and also in Applicant's Co-pending Provisional Application No. 62/032,147 filed Aug. 1, 2014 the disclosures of which are herein incorporated by reference.

For example, a routine that may be responsive to initial motor manifestations and/or tonic activity may include collecting EMG signals over some period of time and integrating the amplitude of collected signals within one or more consecutive or overlapping time windows within that period, and then determining if the integrated amplitude was elevated over a certain threshold for some time as may, for example, be determined if the threshold is met consistently or with some probability over a number of time windows. Levels of EMG signal amplitude may be calculated from signal collected in one or more frequency bands and appropriate filters may be used to isolate one or more target frequency bands. Threshold levels of integrated EMG signal amplitude and/or requirements that a threshold value is maintained for a period of time may, in some embodiments, be set to make that routine responsive to motor manifestations that may be weaker than typically found in a seizure or in a seizure that is likely to be dangerous. Integration time windows may be established to improve detection of relatively weak motor manifestations. For example, in some embodiments, integration time windows for EMG signal collection may be of duration of at least about 20 milliseconds, at least about 50 milliseconds, or at least about 100 milliseconds.

In some embodiments, a threshold level of EMG signal amplitude may be made based on a measurement of a signal amplitude an individual may provide during a voluntary muscle contraction. And, in some embodiments, to capture weak motor manifestations a value of about 2% to about 50% of a maximum voluntary contraction value mny be set.

Also by way of example, a routine that may be selective for clonic phase activity may include determining if a portion of EMG data includes clonic-phase bursts as may be based on fulfilling of a minimum burst width and/or maximum burst width criterion, and if some number of bursts are detected the routine may deemed responsive and clonic-phase activity detected. That is, a routine may count bursts or determine a burst rate and if the number or rate exceeds a threshold a positive response may be logged. In some embodiments, a burst envelope may be generated and the burst envelope may impact a SNR threshold that may be used to identify bursts. For example, with a simple peak detect method, bursts may be qualified by meeting a threshold SNR of about 1.25 to about 20 and by meeting a minimum threshold for burst width of about 25 to about 75 milliseconds and maximum burst width threshold of no greater than about 250 milliseconds to about 400 milliseconds. Bursts may then be counted and a number of bursts or rate of bursts may be determined. For example, a positive routine response may then, for some patients, be triggered if between about 2 to about 6 bursts are measured within a time window of about 1 second or if another suitable number of bursts are counted in some other appropriate time window.

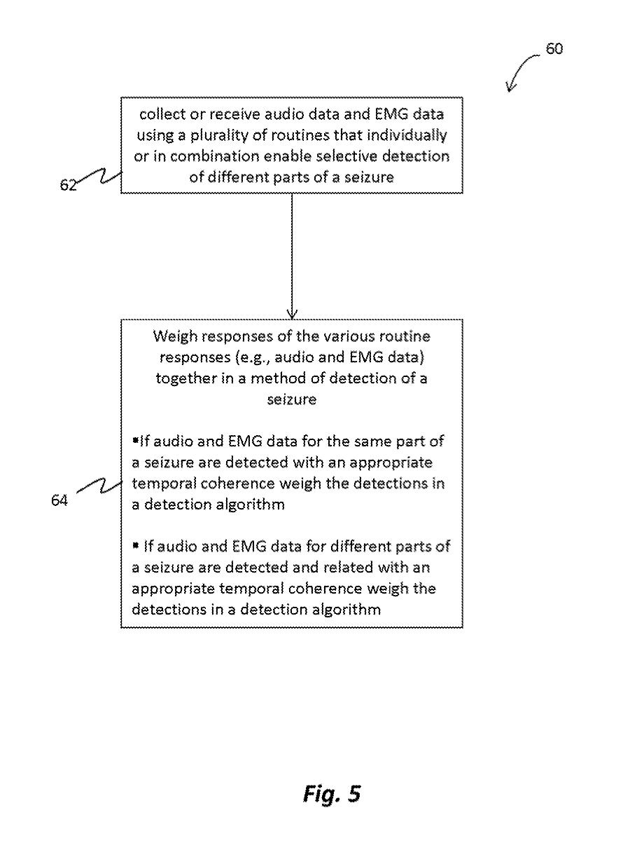

A method 60 of monitoring a patient for seizure characteristics which may include collection and processing or processing of both audio and EMG data is shown in FIG. 5. Like the method 50, the presence of both audio and EMG data may generally increase confidence that a seizure is present. However, in the method 50, it may, in some embodiments, only be required that a seizure event is detected and a corroborating event also detected. For example, one event may be based on EMG data and a corroborating event may be audio data. And, if one event is detected and a corroborating detection made without about 2 minutes the events may be deemed to be corroborated and an alarm may be initiated.

To improve detection efficiency, in the method 60, particular routines are run that individually or in combination may facilitate selective detection of one or more seizure phases or parts. That is, for example, and first considering EMG data, a combination of the aforementioned exemplary routines may be executed. And, if those EMG routines are individually responsive to a given part of a seizure an alarm may be triggered in some patients. Where both routines affirm seizure activity an alarm may also be triggered as confidence in seizure detection and seizure severity may be high. For example, selective detection of clonic activity may be related to adverse effects of a seizure and generally an emergency response may be executed if a tonic-clonic seizure is detected. Where detection of weak motor manifestations or tonic-phase activity is followed by selective detection of clonic-phase activity the pattern of detections may increase confidence that a seizure was detected and may further be used to classify the seizure as a classic tonic-clonic seizure event.

Next, considering audio data, in one routine sound energy may be collected and processed to identify the presence of both high amplitude signals that may be typical of a scream near the start of a seizure and in a second audio detection routine data may be examined for the presence of repetitive patterns that may, for example, be indicative of a person gasping for air as they attempt to deal with or recover from a seizure. In some embodiments, a routine for looking at audio data may also or alternatively identify sounds produced indirectly from a patient struggling during a seizure. For example, a routine may examine audio data for signs that furniture or a sound device is rhythmically moving. Again, where more than one feature of activity is present (e.g., where both routines indicate the presence of signatures of seizure activity) likelihood that a seizure is present is high and an alarm may be triggered. To improve confidence a routine may look for a characteristic lag between the various aspects of audio data. For example, where a repetitive sound pattern is temporally correlated (e.g., separated by an expected time from a scream confidence of detection may be increased. For example, if a scream, commonly indicative of tonic activity, is detected and a repetitive sound pattern is then identified (either front gasping or rhythmic movement of furniture or a sound device) within about 5 to about 45 seconds confidence of seizure detection may be improved. And, the combination may be selectively characterized as a tonic-clonic seizure.

By way of contrast with the method 50, the method 60 may improve detection efficiency by considering in a detection algorithm a temporal relationship between various routine that individually or in combination are selective for one or more parts of a seizure. And, importantly, where two routine for the same part are detected at about the same time the detections may be weighted appropriately. For example, if detections in two routines are made, and where the routines are both selective for times near the start of a seizure the detections may be super-linearly weighted. That is, if the two detections are made and correlated in time contribution of the events to seizure detection may be accordingly adjusted. For example, in some embodiments, the detections may be contribute nonlinearly (or super-additively). In some embodiments, if the detections are made but not correlated in time, the events may still be included in an algorithm to detect a seizure, but only with a reduced weight. Alternatively, it may be required that: temporal coherence between the events is maintained. That is, without being correlated the detections may be discounted. Because the various routines may be correlated with the same part of a seizure, requirements for temporal coherence may be strict and risk of incorrectly identifying to seizure may accordingly minimized.

Referring back to FIG. 5, in a step 62, audio and EMG data may be collected and processed. Alternatively, the method 60 may comprise a method of analyzing sensor data. That is, the sensor data may be collected separately and the method 60 may be used to analyze the data for a seizure event. In the method 60, a plurality of routines may run together. The routines may individually or in combination be selective for one part of a seizure and the method may weigh various detections in a manner based on the expected timing for the various responses as expected in an actual seizure. For example, in some embodiments, each of a first routine and second routine for detection using EMG (including those described above) may be run together (in the step 62) and various algorithms may probe the data for either isolated parts of a seizure and/or for various multi-part seizure events. Likewise, as also shown in the step 62, each of a first and second routine for detection of audio signatures of a seizure may be executed. For example, one routine may analyze collected audio data looking for the occurrence of high amplitude audio data that may indicate the presence of an audible scream as may occur near the start of a seizure and a second audio routine may look for repetitive data indicative of later portions of a seizure or of seizure recovery.

A first routine for EMG detection mar look for tonic phase activity or pre-seizure activity. Where an audible scream is correlated in time with EMG detection of tonic-phase activity the relative detections may be combined in an algorithm for seizure detection. Particularly, in some embodiments, the relative weight of the detections (step 61) may be added in a super-linear manner; that is, in the above example not only were both detections (EMG and audio) made, but the detections were made with temporal coherence in an expected manner and because the parts are often related to the same part of a seizure increase confidence in seizure detection may be particularly high. That is audio and EMG events expected to occur at about the time were made and the signals temporally correlated. In some embodiments, routines for identification of early seizure or tonic phase activity using EMG and routines for detecting an initial high amplitude scream may be deemed temporally correlated and weighted in an algorithm for seizure detection if the events occur within about 1 minute from each.

Likewise, an algorithm may analyze collected audio data looking for the occurrence of repetitive audio data that may, for example, indicate the presence of a patient attempting to regain control of respiration or inducing rhythmic movement of sound, and that may occur after initial manifestations of a seizure. In addition, an algorithm may analyze EMG data using one or more routines selective for clonic-phase activity and/or for EMG data associated with post-seizure recovery. For some patients, the presence of clonic-phase bursts and the presence gasping of air may be highly correlated. And, in some embodiments, routines for identification of clonic phase activity using EMG and routines for detecting repetitive gasps may be deemed temporally correlated and weighted in an algorithm for seizure detection if the events occur within about 30 seconds of each other. Moreover, for some patients trends in periodicity for the aforementioned audio routine and EMG detection routine may be highly correlated. For example, patient motor manifestations as measured in EMG and patient audio responses (e.g., gasping) may be related.

In some embodiments, a threshold level of activation of an acoustic sensor may be based on a level that is some number of standard deviations above a baseline level collected for an acoustic sensor during a non-seizure reference period. Alternatively, in some embodiments, a threshold level of audio activation may be set based on a ratio between an acoustic sensors baseline level and a threshold noise level. For example, a threshold level of an acoustic sensor may be reached upon an increase in acoustic signal of about 10 decibels to about 40 decibels above the acoustic sensors measured baseline level. In other embodiments, a threshold level of activation for an acoustic sensor may be defined based on a sensor reaching a certain decibel level above a standard reference value. An acoustic sensor may, for example, be calibrated against a 0 db signal such as may be typically measured using an external pressure of about 20 micropascals. In some embodiments, a threshold level of activation of an acoustic sensor may be met if the acoustic sensor measures sound at a level exceeding about 50 decibels or about 75 decibels. In some embodiments, a threshold level of audio activation may be high enough that normal speech may not exceed the threshold, but a scream, as may be typical of some patients experiencing a seizure, may exceed a threshold level of activation.

A threshold value of EMG activity may be based on any of various characteristics of EMG activity including for example a T-squared statistical value, presence of amplitude bursts or combinations of EMG characteristics thereof. In some embodiments. EMG signals may be collected for a time period and processed by filtering to select a plurality of frequency bands. For example, an EMG frequency spectrum may be broken up into a number of frequency bands, such as three or more, and one or more characteristics of each frequency band, for example, power content of the band or spectral density at one or more frequencies within the band, may be measured. A measured characteristic for a frequency band may be normalized by its variance and covariance with respect to the characteristic as measured in other frequency bands and resulting normalized values processed to determine one or more T-squared statistical value. A T-squared statistical value may be compared to a reference T-squared statistical value and if the T-squared value exceeds the reference value a threshold condition may be satisfied. In some embodiments, T-squared reference values may be established using one or more reference and/or training periods. For example, a reference T-squared value may be a number of standard deviations from a T-squared baseline obtained while a patient may be resting. In other embodiments, a reference T-squared value may be scaled based on a measurement obtained while a patient may be executing a maximum voluntary contraction and/or may be calculated based on a patients mid-upper arm circumference.

In some embodiments, initiation of an alarm protocol may be dependent upon meeting threshold levels of both audio and EMG activity within a certain period of time. For example, to eliminate false positive detection of a seizure based upon audio signals occurring from non-seizure events, which may also be loud, EMG activation may be required to occur in addition to audio detection, and only if both threshold events occur in an established time period an alarm protocol may be initiated. Temporal correlation of EMG activation and audio activation may be adjusted for individual patient or patient group.

In some embodiments, data from one or more acoustic sensors may be used along with other data from one or more other sensors in a method of seizure detection. For example, audio data may be collected as part of a sub-method in an algorithm configured to periodically probe data from an acoustic sensor and look for periods of high amplitude signals. If detected, the sub-method may increase the value of a register and periodically transfer the registers contents to an accumulation register. An accumulation register may therefore serve as a metric of acoustic activity. An accumulation register may be periodically adjusted (e.g., incremented or decremented) at a desired rate and thereby configured such that only recent acoustic data is held. Therefore, if during a certain time period acoustic activity is high, the accumulation register may tend to increase in value. Other sub-methods, such as more thoroughly described in U.S. patent applications Ser. Nos. 13/275,309 and 13/542,596, may also be operating and may act as sentinels of different characteristics of EMG data. Periodically, as supervisory algorithm may analyze the contents of one or more accumulation registers to determine whether a seizure is likely occurring. If the supervisory algorithm determines that the sum of values or a weighted sum of values in the accumulation registers exceeds a threshold then an alarm protocol may be initiated.

In some embodiments, a plurality of audio sensors may be present in a monitoring region and sounds originating within or near the region may be detected by different sensors. Variation among, the detected signals may be used to spatially filter sound components. For example, spatial filtering of audio data may be used in combination with data associated with an expected or measured position of a patient. For example, sound components likely originating from a location that is spatially distinct from the patient may be discounted or weighted by a factor that decreases the significance of a sound or sound component used in a seizure detection algorithm. In some embodiments, one or more environmental transceivers may be placed in a detection area and as a patient moves the relative position of a patient may be established.

In some embodiments, acoustic data may be analyzed in real-time and integrated in an algorithm for determining whether to initiate an alarm protocol. Analysis of acoustic data may be fully or semi-automated. For example, in some embodiments, acoustic data may include amplitude data or normalized data, and may be integrated into a detection algorithm without the need for interpretation by care-giver personnel. However in some embodiments, audio data may also be sent to a care-giver during or alter a seizure. For example, in some embodiments, audio data or audio data correlating with possible seizure activity may be sent to remote personnel trained to take appropriate action. In some embodiments, data sent to remote personnel may be compressed to reduce transmission bandwidth or processed to encourage efficient analysis by care-giver personnel. For example, audio and/or EMG data may be suitably compressed so that the information may be readily scrolled through during analysis.

In some embodiments, detection of a seizure or possible seizure related event may trigger automatic transmission of EMG and audio data to a remote monitoring facility. For example, if an alarm is triggered data proceeding and after the event may be sent for review. In some embodiments. EMG data may be decimated to reduce the size of the file, but not decimated so much as to lose visible quality. Reduction of the file may, for example, make it more responsive when manipulating the data from a local computer with internet service. A caregiver viewing the data on a local computer may then select to view/listen to any portion of the transmitted data. In one embodiment, a five minute interval on either side of art expected event (e.g., 10 minutes of data) may be sent and/or uploaded for review. A care-giver viewing the data an a local computer may select to view/listen to the entire ten minutes or select on a series of buttons labeled 1-10 to view/listen at a particular 1 minute segment. The software may be configured such that a selected portion of EMG data may scroll across the screen at a rate such that associated audio data (e.g., data collected at the same time as the EMG data) is simultaneously heard.

Although the disclosed method and apparatus and their advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition, or matter, means, methods and steps described in the specification. Use of the word “include,” for example, should be interpreted as the word “comprising,” would be, i.e., as open-ended. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods or steps.

1. A method of detecting seizures with motor manifestations comprising:
receiving EMG data for a first period of time;
receiving audio data from said first period of time;
determining for said first period of time whether said EMG data meets a first EMG data threshold condition and/or if said audio data meets a first audio data threshold condition;
receiving EMG and audio data for a second period of time if either or both of said first EMG threshold condition and/or said first audio data threshold condition is met; and
determining for said second period of time whether either or both of said EMG data meets a second EMG data threshold condition and/or if said audio data meets a second audio data threshold condition;
initiating an alarm if, during said second time period, either or both of said second EMG threshold condition and/or said second audio data threshold condition is met.

2. The method of claim 1 wherein meeting said first audio data threshold condition includes reaching a threshold level of audio signal amplitude followed by a sustained period of lower amplitude audio data.

3. The method of claim 1 wherein meeting said first audio data threshold condition includes reaching an audio signal amplitude of at least about 50 decibels to about 75 decibels followed by a decreased audio signal, the decreased audio signal lasting for at least about 5 seconds.

4. The method of claim 1 wherein meeting said first audio data threshold condition includes detection of one or more parts of audio data that repeat within a time period of about 0.2 to about 2 seconds.

5. The method of claim 4 wherein said one or more parts of audio data that repeat are selected from a group of parts including a threshold amplitude of audio data, a threshold local maximum value in amplitude, a local maximum value in amplitude followed by a sustained decrease in amplitude of the audio data, and a data point in a pattern of audio data identified by pattern recognition.

6. The method of claim 4 wherein said one or more parts of audio data that repeat include a portion of audio data qualified by regression analysis as being suitably similar to a model of portion of audio data.

7. The method of claim 6 wherein said model portion of audio data is derived from recordings of patient's gasping for air during an inhalation part of a recorded seizure.

8. The method of claim 4 wherein the one or more parts of audio data that repeat repeat at least about 4 to about 10 times to meet said first audio data threshold condition.

9. The method of claim 1 wherein said second time period extends for a period of time of about 2 minutes from when said first threshold condition is met.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A method of monitoring a patient for seizure activity comprising:
receiving audio data and selecting from the received audio data a subset of audio data that may be indicative of a seizure;
transmitting the subset of audio data to a remote caregiver trained to interpret if the data is indicative of a seizure; and
triggering an alarm response if said audio data indicates that a seizure may be present.

22. The method of claim 21 wherein said subset of audio data includes audio data identified by a pattern recognition program where an identified pattern repeats over a time period of about 0.2 to about 2 seconds, the identified pattern being present at least about 4 to about 10 times.

23. The method of claim 21 further comprising detection of EMG signal data;
wherein said subset of audio data comprises data following detection of an increase in EMG signal amplitude.

24. The method of claim 21 wherein the increase in EMG signal amplitude is an increase in EMG signal of about 2% to about 50% of a maximum voluntary contraction.

25. A method of detecting seizures with motor manifestations comprising:
collecting audio data over a plurality of time periods using one or more acoustic sensors;
calculating one or more values of a characteristic of the collected acoustic data for each of a number of time periods among said plurality of time periods;
analyzing whether a value of the characteristic meets one or more criteria;
calculating one or more times between consecutive values that meet said one or more criteria;
determining whether said one or more times meet a periodicity condition for a patient experiencing a seizure; and
integrating the determination of periodicity in a decision about whether to initiate an alarm protocol.

26. The method of claim 25 wherein said characteristic includes an acoustic amplitude; and
wherein said criteria includes whether said acoustic amplitude is a local maximum value that is greater than a threshold amplitude value.
